(19) United States
(12) Patent Application Publication
(10) Pub. No.: US 2009/0024002 A1
(43) Pub. Date: Jan. 22, 2009

(54) MONITORING DEVICE FOR PATIENT AND MONITORING SYSTEM AND METHOD USING SAME

(75) Inventors: PING-TUAN SONG, Shenzhen (CN); SHIH-FANG WONG, Tu-Cheng (TW)

Correspondence Address: PCE INDUSTRY, INC.;ATT. CHENG-JU CHIANG, 458 E. LAMBERT ROAD, FULLERTON, CA, 92835, US

(73) Assignees: HONG FU JIN PRECISION INDUSTRY (shenZhen) Co., LTD, Shenzhen City (CN); HON HAI PRECISION INDUSTRY CO., LTD., Tu-Cheng (TW)

(21) Appl. No.: 11/954,226

(22) Filed: Dec. 12, 2007

(30) Foreign Application Priority Data
Jul. 17, 2007 (CN) 200710201107.2

(51) Int. Cl. A61B 5/00

(52) U.S. Cl. 600/300

(57) ABSTRACT

A monitoring system includes a mobile communication device, a base station, and a monitoring device. The mobile communication device is used for detecting physiological parameters of a patient and transmitting first wireless signals carrying the physiological parameters and an identification of the patient. The monitoring device is used for receiving the first wireless signals through the base station, and searching for addresses and telephone numbers of hospitals and pharmacies, and transmitting second wireless signals carrying a doctor input, the addresses, and the telephone numbers to the mobile communication device through the base station. A monitoring method is also provided.

### BACKGROUND

1. Field of the Invention

The present invention generally relates to medical systems, and more particularly to a monitoring device for patient, and a monitoring system and a monitoring method using the same.

2. Description of Related Art

Nowadays, many medical systems are provided for patients. Among the medical systems, monitoring systems are widely used to help doctors to obtain physiological parameters of patients. A monitoring system can be installed in a patient's home to monitor the patient. The monitoring system can transmit health information of the patient to a hospital and receive some treatment advices therefrom via the Internet or telephone. However, the range of the monitoring systems is usually within the confines of the patient's home. If the patient walks outside of his home, the monitoring system may not be able to monitor the patient.

Therefore, improvements for a monitoring device, a monitoring system and a monitoring method for monitoring patients are needed in the industry to address the aforementioned deficiency.

### SUMMARY

A monitoring system includes a mobile communication device, a base station, and a monitoring device. The mobile communication device is used for detecting physiological parameters of a patient and transmitting first wireless signals carrying the physiological parameters and an identification of the patient. The monitoring device is used for receiving the first wireless signals through the base station, and searching for addresses and telephone numbers of hospitals and pharmacies, and transmitting second wireless signals carrying a doctor input, the addresses, and the telephone numbers to the mobile communication device through the base station. A monitoring method is also provided.

Other advantages and novel features of the present invention will become more apparent from the following detailed description of preferred embodiment when taken in conjunction with the accompanying drawings.

### BRIEF DESCRIPTION OF THE DRAWINGS

FIG. 1 is a schematic diagram showing a monitoring system in accordance with an exemplary embodiment;

FIG. 2 is a process flow diagram illustrating a monitoring method in accordance with an exemplary embodiment; and

FIG. 3 is a schematic, block diagram showing the monitoring system of FIG. 1.

### DETAILED DESCRIPTION OF THE EMBODIMENTS

Reference will now be made to the drawings to describe a preferred embodiment of the present monitoring device, a preferred embodiment of the present monitoring system, and a preferred embodiment of the present monitoring method.

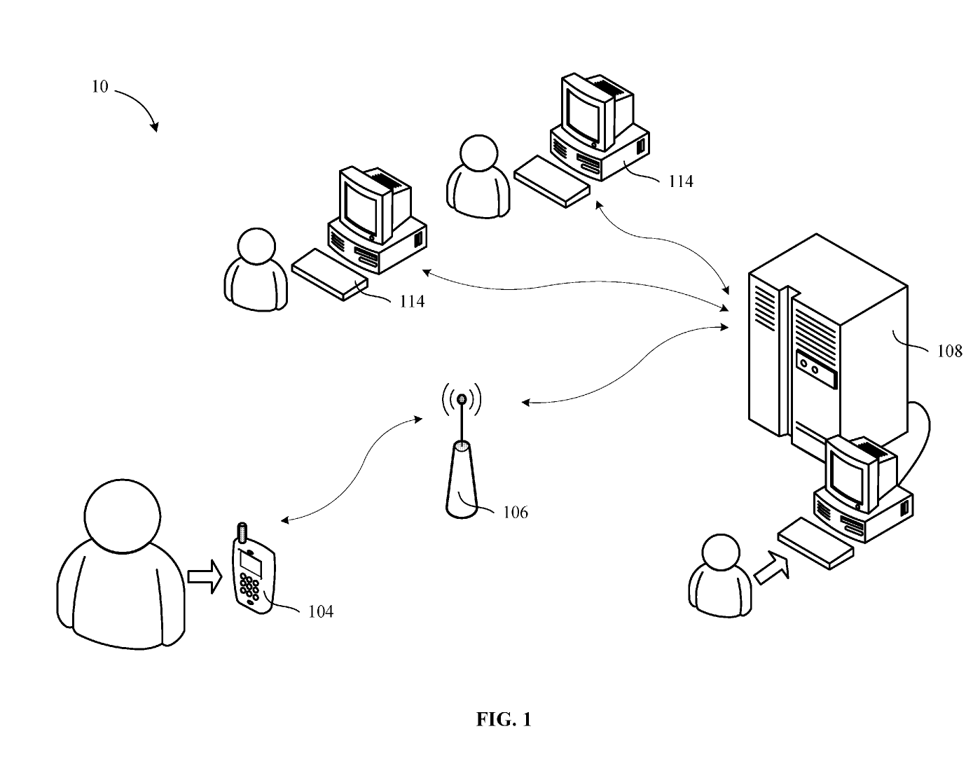

Referring to FIG. 1, a monitoring system 10 in accordance with an exemplary embodiment is used for monitoring physiological parameters, such as blood pressure or heart rate, of patients. The monitoring system 10 includes a mobile communication device 104, a base station 106, a monitoring device 108, and a plurality of service terminals 114. The communication device 104 is used by a patient to self-detect physiological parameters, and transmitting first wireless signals carrying the physiological parameters and an identification of the patient to the base station 106. The base station 106 is used for connecting the mobile communication device 104 and the monitoring device 108 wirelessly, and transmitting information between the mobile communication device 104 and the monitoring device 108.

The monitoring device 108 is positioned in a hospital for receiving the physiological parameters through the base station 106, and is used for receiving a doctor input to temporary treat the patient. The doctor input may include names of medicines, treatment methods, and so on. In addition, the monitoring device 108 is also used for searching for addresses and telephone numbers of other hospitals and pharmacies that are near a current location of the patient, and transmitting second wireless signals carrying the doctor input, the addresses, and the telephone numbers to the mobile communication device 104 through the base station 106. The service terminals 114 are positioned in the other hospitals and the pharmacies for updating the addresses and the telephone numbers.

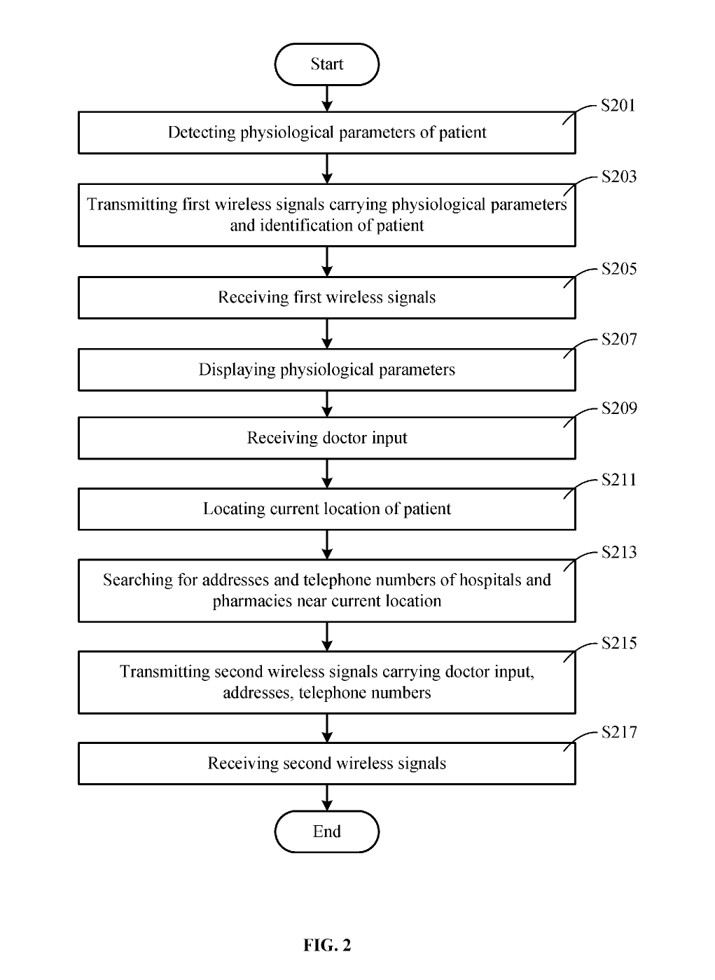

Referring to FIG. 2, a monitoring method in accordance with an exemplary embodiment is used for monitoring a patient. A procedure of the monitoring method includes the following steps.

Step S201, the communication device 104 detects physiological parameters of the patient.

Step S203, the mobile communication device transmits first wireless signals carrying the physiological parameters and an identification of the patient to the monitoring device 108 through the base station 106.

Step S205, the monitoring device 108 receives the first wireless signals and verifies the patient's identification.

Step S207, the monitoring device 108 displays the physiological parameters to doctors.

Step S209, the monitoring device 108 receives a doctor input.

Step S211, the monitoring device 108 locates a current location of the mobile communication device 104 in a service area of the base station 106.

Step S213, the monitoring device 108 searches for addresses and telephone numbers of other hospitals and pharmacies near the current location of the mobile communication device 104.

Step S215, the monitoring device 108 transmits second wireless signals carrying the doctor input, the addresses, and the telephone numbers to the mobile communication device 104 through the base station 106.

Step S217, the mobile communication device 104 receives the second wireless signals and displays the doctor input, the addresses, and the telephone numbers to the patient.

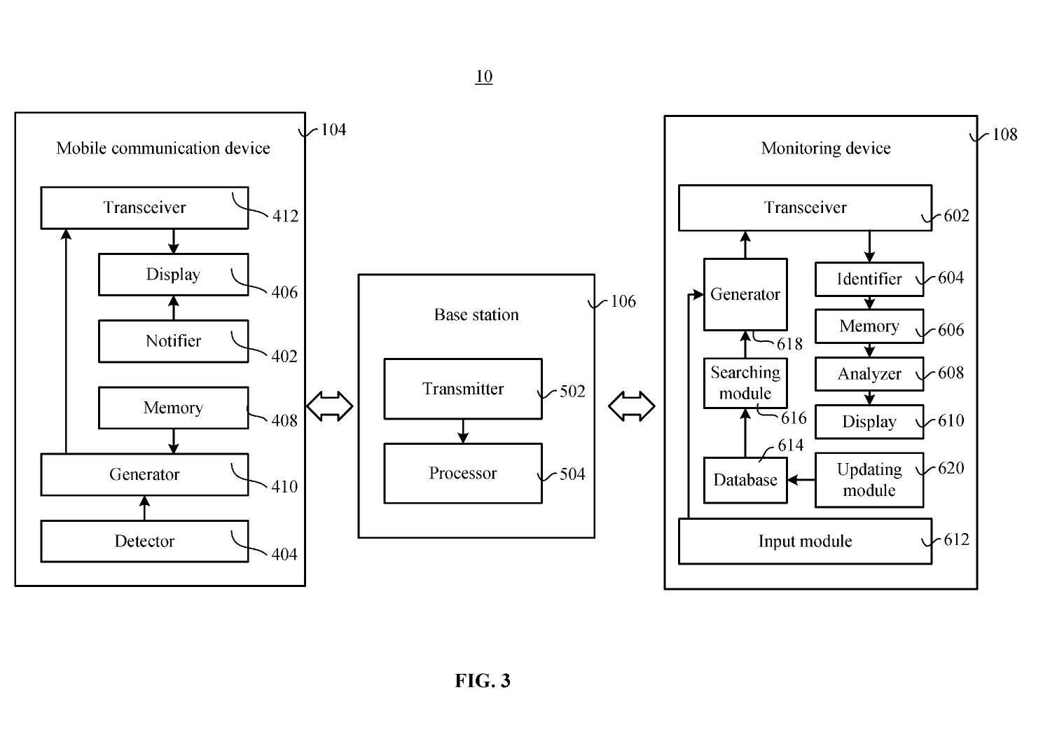

Referring to FIG. 3, a block diagram of the monitoring system 10 is illustrated. In the monitoring system 10, the mobile communication device 104 includes a notifier 402, a detector 404, a display 406, a memory 408, a generator 410, and a transceiver 412. The notifier 402 is used for notifying the patient to detect physiological parameters by playing a sound or vibrating. The detector 404 is used for detecting the physiological parameters. The display 406 is used for displaying the physiological parameters, the doctor input, the addresses, and the telephone numbers. The memory 408 is used for storing the patient identification. The generator 410 is used for generating the first wireless signals according to the physiological parameters and the patient's identification. The transceiver 412 is used for transmitting the first wireless signals to the base station 106, and receiving the doctor input, the addresses, and the telephone numbers from the base station 106.

The base station 106 includes a transmitter 502 and a processor 504. The transmitter 502 is used for transmitting information between the mobile communication device 104 and the monitoring device 108. The processor 502 is used for adding an identification of the base station 106 to the first wireless signals.

The monitoring device 108 includes a transceiver 602, an identifier 604, a memory 606, an analyzer 608, a display 610, an input module 612, a database 614, a searching module 616, a generator 618, and a updating module 620.

The transceiver 602 is used for receiving the first wireless signals from the base station 106, and transmitting second wireless signals carrying the doctor input, the addresses, and the telephone numbers to the base station 106. The identifier 604 is used for verifying the identification of the patient. The memory 606 is used for storing a case file of the patient. The analyzer 608 is used for analyzing a current health of the patient according to the physiological parameters and the case file. The display 610 is used for displaying the physiological parameters and analyzed results. The input module 612 is used for receiving the doctor input including names of medicines, treatment methods, and so on.

The database 614 is used for storing the addresses and the telephone numbers of other hospitals and pharmacies. The searching module 616 is used for locating the mobile communication device 104 in the service area of the base station 106, and searching the database 614 for the addresses and the telephone numbers according to the identification of the base station 106. The generator 618 is used for generating the second wireless signals according to the doctor input, the addresses, and the telephone numbers. The updating module 620 is used for receiving new addresses, and new telephone numbers from the service terminals 114 and updating the information stored in the database 614.

In operation, the notifier 402 notifies the patient to detect physiological parameters. The detector 404 is enabled to detect the physiological parameters by the patient. The display 406 displays the physiological parameters and the generator 410 generates the first wireless signals according to the physiological parameters and the patient's identification stored in the memory 408. The transceiver 412 transmits the first wireless signals to the base station 106. The processor 502 of the base station 106 adds the identification of the base station 106 to the first wireless signals, and then the transmitter 502 transmits the first wireless signals to the monitoring device 108.

The transceiver 602 of the monitoring device 108 receives the first wireless signals from the base station 106. The identifier 604 verifies the identification of the patient carried in the first wireless signals. The analyzer 608 analyzes the current health of the patient according to the physiological parameters and the case file stored in the memory 606. The display 610 displays the physiological parameters and the analyzed results. The input module 612 receives the doctor input. The searching module 616 locates the current location of the mobile communication device 104, and searches the database 614 for the addresses and the telephone numbers according to the identification of the base station 106. The generator 618 generates the second wireless signals according to the doctor input, the addresses, and the telephone numbers. The updating module 620 receives the new addresses and the new telephone numbers, and updates the database 614.

The transceiver 602 transmits the second wireless signals to the base station 106. The transmitter 502 of the base station 106 transmits the second wireless signals to the mobile communication device 104. The transceiver 412 receives the second wireless signals. The display 406 displays the doctor input, the addresses, and the telephone numbers carried in the second wireless signals.

As mentioned above, the monitoring system 10 uses the mobile communication device 104 to detect the patient's physiological parameters, and uses the monitoring device 108 to analyze the physiological parameters and transmit help information to the mobile communication device 104. Therefore, no matter where the patient goes, the monitoring system 10 can monitor and provide feedback that may help him.

It is to be understood, however, that even though numerous characteristics and advantages of the present invention have been set forth in the foregoing description, together with details of the structure and function of the invention, the disclosure is illustrative only, and changes may be made in detail, especially in matters of shape, size, and arrangement of parts within the principles of the invention to the full extent indicated by the broad general meaning of the terms in which the appended claims are expressed.

What is claimed is:

1. A monitoring system comprising:
a mobile communication device for detecting physiological parameters of a patient and transmitting first wireless signals carrying the physiological parameters and identification of the patient; and
a monitoring device for receiving the first wireless signals through a base station, and searching for addresses and telephone numbers of hospitals and pharmacies, and transmitting second wireless signals carrying a doctor input, the addresses, and the telephone numbers to the mobile communication device through the base station.

2. The monitoring system according to claim 1, further comprising a plurality of service terminals positioned in the hospitals and the pharmacies for updating the addresses and the telephone numbers.

3. The monitoring system according to claim 1, wherein the mobile communication device comprises a detector for detecting the physiological parameters.

4. The monitoring system according to claim 1, wherein the mobile communication device comprises a notifier for notifying the patient to check the physiological parameters.

5. The monitoring system according to claim 1, wherein the mobile communication device comprises a display for displaying the physiological parameters, the doctor input, the addresses, and the telephone numbers.

6. The monitoring system according to claim 1, wherein the mobile communication device comprises a generator for generating the first wireless signals according to the physiological parameters and the identification of the patient.

7. The monitoring system according to claim 1, wherein the mobile communication device comprises a transceiver for transmitting the first wireless signals and receiving the second wireless signals.

8. The monitoring system according to claim 1, wherein the base station comprises a processor for adding an identification of the base station to the first wireless signals.

9. The monitoring system according to claim 8, wherein the base station further comprises a transmitter for transmitting the first wireless signals and the second wireless signals.

10. A monitoring device comprising:
a transceiver for receiving first wireless signals from a base station and for transmitting second wireless signals, the first wireless signals carrying physiological parameters of a patient and a first identification of the patient and a second identification of the base station;
a display for displaying the physiological parameters;
an input module for receiving a doctor input; and
a generator for generating the second wireless signals carrying the doctor input.

11. The monitoring device according to claim 10, further comprising:
an identifier for verifying the first identification;
a memory for storing a case file of the patient;
an analyzer for analyzing a current health of the patient according to the physiological parameters and the case file.

12. The monitoring device according to claim 11, wherein the display is for displaying analyzed results.

13. The monitoring device according to claim 10, further comprising a database for storing addresses and telephone numbers of hospitals and pharmacies.

14. The monitoring device according to claim 13, further comprising a searching module for determining the current location of the patient in service area of the base station, and searching the database for addresses and telephone numbers of hospitals and pharmacies near the current location according to the second identification.

15. The monitoring device according to claim 14, wherein the second wireless signals further carry the addresses and the telephone numbers of the hospitals and the pharmacies near the current location.

16. The monitoring device according to claim 13, further comprising an updating module for receiving new address and new telephone numbers and updating the database.

17. A monitoring method comprising:
detecting physiological parameters of a patient;
transmitting first wireless signals carrying the physiological parameters and an identification of the patient;
receiving the first wireless signals;
verifying the identification;
displaying the physiological parameters;
receiving a doctor input; and
transmitting second wireless signals carrying the doctor input.

18. The monitoring method according to claim 17, further comprising:
determining a current location of the patient;
searching for addresses and telephone numbers of hospitals and pharmacies near the current location.

19. The monitoring method according to claim 18, wherein the second wireless signals carry the addresses and the telephone numbers.
